(19) United States
(12) Patent Application Publication
(10) Pub. No.: US 2002/0146375 A1
(43) Pub. Date: Oct. 10, 2002

(54) Cosmetic or pharmaceutical lecithin-containing gels or low viscosity lecithin-containing O/W microemulsions

(76) Inventors: Jorg Schreiber, Hamburg (DE); Florian Wolf, Hamburg (DE); Delphine Croizet, Jarnac (FR)

Correspondence Address: Kurt G. Briscoe, Norris McLaughlin & Marcus, P.A., 30th Floor, 220 East 42nd Street, New York, NY, 10017, US

(21) Appl. No.: 09/894,771

(22) Filed: Jun. 28, 2001

(30) Foreign Application Priority Data
Dec. 22, 1998 (DE) 198 59 427.5

(51) Int. Cl. A61K 7/42; A61K 7/075; A61K 7/08

(52) U.S. Cl. 424/59; 424/70.23

(57) ABSTRACT

A gel or low-viscosity transparent or translucent microemulsion of the oil-in-water type, comprising at least one phospholipid and at least one oil-in-water emulsifier.

The present invention relates to phospholipid-containing gels or microemulsions of the oil-in-water type, to processes for their preparation and to their use for cosmetic or pharmaceutical purposes. In particular, they are applied topically.

Cosmetic skincare is primarily to be understood as meaning that the natural function of the skin as a barrier against environmental influences (e.g. dirt, chemicals, microorganisms) and against the loss of endogenous substances (e.g. water, natural fats, electrolytes) is strengthened or restored.

If this function is impaired, increased resorption of toxic or allergenic substances or attack by microorganisms may result, leading to toxic or allergic skin reactions.

Another aim of skincare is to compensate for the loss by the skin of lipids and water caused by daily washing. This is particularly important if the natural regeneration ability is inadequate. Furthermore, skincare products should protect against environmental influences, in particular against sun and wind, and delay skin aging.

Medicinal compositions generally comprise one or more medicaments in an effective concentration. For the sake of simplicity, in order to differentiate clearly between cosmetic and medicinal use and corresponding products, reference is made to the legal provisions of the Federal Republic of Germany (e.g. Cosmetics Directive, Foods and Drugs Act).

Cosmetic or dermatological preparations are frequently in the form of finely dispersed multiphase systems in which one or more fat or oil phases are present in addition to one or more water phases. Of these systems, in turn, the actual emulsions are the most widespread.

In simple emulsions, finely disperse droplets of one phase (water droplets in W/O emulsions or lipid vesicles in O/W emulsions), surrounded by an emulsifier shell, are present in a second phase. The droplet diameters of customary emulsions are in the range from about 1 μm to about 50 μm. Such “macroemulsions” are, without further coloring additives, milky white in color and opaque. Finer “macroemulsions”, whose droplet diameters are in the range from about 10⁻¹ μm to about 1 μm are, again without coloring additives, bluish white in color and nontransparent.

Only micellar and molecular solutions having particle diameters of less than about 10⁻² μm appear clear and transparent.

By contrast, the droplet diameters of transparent or translucent microemulsions are in the range from about 10⁻² μm to about 10⁻¹ μm. Such microemulsions are mostly low-viscosity. The viscosity of many microemulsions of the O/W type is comparable with that of water.

An advantage of microemulsions is that active ingredients can be present in more finely disperse form in the disperse phase than in the disperse phase of “macroemulsions”. A further advantage is that they are sprayable as a result of the low viscosity. If microemulsions are used as cosmetics, corresponding products are characterized by high cosmetic elegance.

A disadvantage of the microemulsions of the prior art is that a high content of one or more emulsifiers must always be used since the low droplet size brings about a large interface between the phases, which usually has to be stabilized by emulsifiers.

The use of customary cosmetic emulsifiers is in itself safe. Nevertheless, emulsifiers, like ultimately any chemical substance, may in individual cases cause allergic reactions or reactions based on user hypersensitivity.

For example, it is known that certain photodermatoses are triggered by said emulsifiers, but also by various fats, and simultaneous exposure to sunlight. Such photodermatoses are also called “Mallorca acne”. One object of the present invention was therefore to develop sunscreen products.

Thus, the present invention relates, as a particular embodiment, to cosmetic and dermatological light protection preparations, in particular skincare cosmetic and dermatological light protection preparations.

The harmful effects of the ultraviolet part of solar radiation on the skin is generally known. While rays having a wavelength of less than 290 nm (the UVC region) are absorbed by the ozone layer in the earth's atmosphere, rays in the range between 290 nm and 320 nm, the UVB region, cause erythema, simple sunburn or even more or less serious burns.

The erythema activity maximum of sunlight is stated as the relatively narrow range around 308 nm.

Numerous compounds are known for protecting against UVB radiation; these are mostly derivatives of 3-benzylidene camphor, of 4-aminobenzoic acid, of cinnamic acid, or salicylic acid, of benzophenone and also of 2-phenylbenzimidazol.

For the range between about 320 nm and about 400 nm, the UVA region, it is also important to have available filter substances since rays of that region can also cause damage. For example, it has been proven that UVA radiation leads to damage of the elastic and collagenous fibers of connective tissue, causing premature aging of the skin, and that it is to be regarded as a cause of numerous phototoxic and photoallergic reactions. The harmful effect of UVB radiation can be intensified by UVA radiation.

UV radiation can, however, also lead to photochemical reactions, in which case the photochemical reaction products then intervene in the skin's metabolism.

In order to prevent these reactions, antioxidants and/or free-radical scavengers can additionally be incorporated into the cosmetic or dermatological formulations.

Most of the inorganic pigments which are known for use in cosmetics for protecting the skin against UV rays are UV absorbers or UV reflectors. These pigments are oxides of titanium, zinc, iron, zirconium, silicon, manganese, aluminum, cerium and mixtures thereof, and also modifications.

Because of their good sprayability, microemulsions are also suitable for other cosmetic [lacuna] dermatological applications, for example deodorants, meaning that the present invention relates, in a particular embodiment, to microemulsions as a basis for cosmetic deodorants.

Cosmetic deodorants serve to eliminate body odor which arises when fresh perspiration, which is in itself odorless, is decomposed by microorganisms. Customary cosmetic deodorants are based on different active principles.

In antiperspirants, the formation of perspiration can be reduced by astringents—chiefly aluminum salts such as aluminum hydroxychloride (aluminum chlorhydrate).

By using antimicrobial substances in cosmetic deodorants it is possible to reduce the bacterial flora on the skin. In an ideal case, only the odor-causing microorganisms would be effectively reduced. The flow of perspiration itself is not influenced by this, and in an ideal case only microbial decomposition of the perspiration is temporarily stopped.

The combination of astringents with antimicrobial substances in one and the same composition is also customary.

Deodorants should satisfy the following conditions:

1) They should effect reliable deodorization.

2) The natural biological processes of the skin must not be impaired by the deodorant.

3) The deodorant must be harmless in the event of an overdose or other use which is not as specified.

4) They should not become concentrated on the skin following repeated application.

5) They should be easy to incorporate into customary cosmetic formulations.

Liquid deodorants, for example aerosol sprays, roll-ons and the like, and also solid preparations, for example deodorant sticks, powders, powder sprays, intimate cleansing compositions etc. are known and customary.

The use of microemulsions as a base for deodorizing or antiperspirant preparations are also known. Their relatively high content of emulsifiers, together with the described disadvantages, has hitherto been a shortcoming which has been in need of remedying.

A further object of the present invention was therefore to develop preparations which are suitable as bases for cosmetic deodorants or antiperspirants and do not have the disadvantages of the prior art.

It was also an object of the invention to develop cosmetic bases for cosmetic deodorants which are characterized by good skin compatibility.

In addition it was an object of the present invention to make available products based on microemulsions having the broadest possible application diversity. For example, bases for preparation forms such as cleansing emulsions, face- and bodycare preparations were to be provided, but also decidedly medicinal-pharmaceutical administration forms, for example preparations against acne and other skin phenomena.

In a particular embodiment, the invention therefore relates to cleansing emulsions, in particular face cleansing emulsions, preferably make-up removers, for example eye make-up removers.

Such preparations are known per se. They are usually mixtures of cosmetic oils or aqueous preparations of surface-active substances, the function of which is to solubilize the foreign substance or the make-up substance and remove it from the skin.

Water resistant eye make-up, for example, mascara, can only be removed satisfactorily with aqueous-based make-up removers containing specific surfactants. However, the surfactants often only have limited physiological compatibility. When such substances come into contact with the mucous membrane, in particular the mucous membrane of the eye, they lead to irritations which manifest themselves, for example, in a reddening of the eyes. Reactions of this type are typical of surfactant-containing products.

An object of the present invention was therefore to remedy such problems.

The present invention relates in a further embodiment to hair cosmetic preparations. In particular, the present invention relates to hair cosmetic preparations for the care of hair and the scalp. In a preferred embodiment, the present invention relates to preparations which serve to strengthen individual hairs and/or impart hold and body to the hairstyle overall.

Roughly speaking, human hair can be divided into the living part, the hair root, and the dead part, the hair shaft. The hair shaft in turn comprises the medulla which, however, as a result of evolution, has become insignificant for modern man and has receded, and in cases of thin hair, is often absent entirely, and also the cortex surrounding the medulla and the cuticula which encloses the totality of medulla and cortex.

The cuticula in particular, but also the keratinous region between the cuticula and cortex, as the outer sheath of the hair, are exposed to particular demands as a result of environmental influences, as a result of combing and brushing, but also as a result of hair treatment, in particular the coloring and shaping of hair, e.g. permanent waving processes.

If the stress is particularly aggressive, for example bleaching with oxidizing agents such as hydrogen peroxide, in which the pigment distributed within the cortex are destroyed by oxidation, the inside of the hair can also be affected. If human hair is to be colored permanently, in practice only oxidizing hair coloring processes are suitable. During the oxidative coloring of hair, the dye chromophors are formed as a result of the reaction of precursors (phenols, aminophenols, and less frequently also diamines) and bases (in most cases p-phenylenediamine) with the oxidizing agent, in most cases hydrogen peroxide. Hydrogen peroxide concentrations of about 6% are usually used for this.

It is usually assumed that in addition to the coloring action, a bleaching action also takes place as a result of the hydrogen peroxide. In oxidatively colored human hair, as in the case of bleached hair, microscopic holes are detectable at the points where melanin granules were present. The fact is that the oxidizing agent hydrogen peroxide can react not only with the dye precursors, but also with the hair substance and as a result cause damage to the hair under certain circumstances.

Washing the hair with aggressive surfactants can also stress the hair, and at least reduce its appearance or the appearance of the hairstyle overall. For example, certain water-soluble constituents of hair (e.g. urea, uric acid, xanthine, keratin, glycogen, citric acid and lactic acid) can be leached out as a result of hair washing.

For these reasons, some haircare cosmetics which are intended to be rinsed out of the hair again once they have acted, and some of those which remain on the hair have been used for a relatively long time. The latter can be formulated such that they not only serve to care for the individual hairs, but also improve the appearance of the hairstyle overall, for example by imparting more body to the hair, fixing the hairstyle over a relatively long period or improving its ease of styling.

By using quaternary ammonium compounds, for example, the combability of the hair can be decisively improved. Such compounds attach to the hair and are often still detectable on the hair after the hair has been washed a number of times.

However, the prior art has lacked active ingredients and preparations which satisfactorily care for damaged hair. Preparations which are intended to give body to the hairstyle have also often proven to be inadequate, or they were at least unsuitable for use as haircare preparations. The preparations of the prior art which fix the hairstyle generally comprise, for example, viscous constituents which run the risk of giving rise to a feeling of tackiness, which often has to be compensated for by skillful formulation.

An object was therefore also to overcome these the disadvantages of the prior art.

Finally, it was also the aim to in principle open up the way for emulsions which can be used internally, for example for the parental administration of pharmaceutical active ingredients and for parental feeding by the present invention.

A particular object of the present invention was to make available finely disperse preparations of the oil-in-water type with the lowest possible emulsifier content which do not have the disadvantages of the prior art and which can be used for a very wide variety of cosmetic and/or dermatological applications, for example the uses described above. A further object of the invention was to enrich the limited range of finely disperse preparations of the oil-in-water type of the prior art.

It is known per se that hydrophilic emulsifiers, namely polyethoxylated and polypropoxylated emulsifiers, change their solubility behavior from water-soluble to fat-soluble with increasing temperature. An indicator of the hydrophilicity of a given emulsifier is its HLB value.

The definition of the HLB value is given, for polyol fatty acid esters, by the relationship

HLB=20*(1−S/A) (formula I)

For a group of emulsifiers whose hydrophilic fraction consists only of ethylene oxide units, the following relationship applies

HLB=E/5 (formula II)

where S=saponification number of the ester,

A=acid number of the recovered acid

E=mass fraction of ethylene oxide (in %) of the overall molecule.

Emulsifiers with HLB values of 6-8 are generally W/O emulsifiers, and those with HLB values of 8-18 are generally O/W emulsifiers.

Literature: “Kosmetik - Entwicklung, Herstellung und Anwendung kosmetischer Miffel” [Cosmetics - development, preparation and use of cosmetic compositions]; W. Umbach (Ed.), Georg Thieme Verlag 1988.

The temperature range within which the emulsifiers change their solubility is referred to as the phase inversion temperature range. Within this specification, the abbreviation “PIT” will also be used for the phase inversion temperature range.

The change in this solubility behavior is evident, as is known, from the fact that a mixture of water, oil and O/W emulsifiers which produces an O/W emulsion below the PIT after stirring, is brought to a temperature above the PIT, typically about 70-90° C., can pass through the state of a microemulsion as intermediate stage in order finally to give a W/O emulsion above the PIT. If this emulsion is cooled, an O/W emulsion is again obtained, although it has a droplet size of up to 200 nm and is in the range between a microemulsion and a fine macroemulsion.

However, microemulsions of the prior art prepared in this way have the disadvantage that firstly the droplet size is still very high, and the emulsion is opaque white bluish at room temperature and/or a high content of one or more emulsifiers is still necessary.

It is also disadvantageous that although microemulsions prepared in this way can be virtually transparent at high temperature, i.e. for example in the PIT, they become nontransparent again when the temperature is reduced to room temperature.

It was therefore also the aim to remedy these shortcomings.

It was a particular object of the present invention to make available low-viscosity preparations based on finely disperse systems of the oil-in-water type having the lowest possible emulsifier content which do not have the disadvantages of the prior art and which can be used for a very wide variety of cosmetic and/or dermatological applications, for example the uses described above. It was a further object of the invention to enrich the limited range of low-viscosity preparations based on finely disperse lecithin-containing systems of the oil-in-water type of the prior art.

Lecithin-containing microemulsions for cosmetic, pharmaceutical, parenteral applications are known from the literature. Droplet sizes below 100 nm are achieved by high-pressure homogenization of corresponding macroemulsions. A disadvantage is that high shear forces on the droplets arise and metal abrasion takes place, which can only be removed from the corresponding administration forms with difficulty. In addition, ultrasound can also be used to prepare corresponding microemulsions. It is disadvantageous that these processes are expensive due to the high input of energy.

Sometimes it is disadvantageous in the case of known O/W microemulsions which do not contain phospholipids that they do not always have entirely satisfactory care effects (skin moisturizing, skin roughness reduction, skin flakiness reduction).

In addition, microemulsions containing lecithin are obtained in the presence of high concentrations of short-chain alcohols, alkanediols, amines, which are unsuitable for cosmetic, pharmaceutical and parenteral applications.

High-pressure homogenization or ultrasound for the preparation of parenteral emulsions, for cosmetic or pharmaceutical applications are described in the literature. Int. J. Pharm. 163, 1998, 81; J. Pharm. Belg. 52, 1997, 110; J. Pharm. Sci. 82, 1993, 1069; J. Pharm. Sci. 83, 1994, 72; Parf. and Kosmet. 10, 1994, 652; 3, 1995, 152; Pharm. Res. 12,1995,1273; SÖFW 9,1994, 530.

Phospholipid O/W microemulsions with cosolvents such as short-chain alcohols (propanol, butanol, ethanol, isopropanol, sec-butanol, tert-butanol, n-pentanol); alkanediols, short-chain alkyl ethers or amines are described in the literature. Int. J. Pharm. 125, 1995, 107; Int. J. Pharm. 111, 1994, 63; Int. J. Pharm. 161, 1993, 161; Int. J. Pharm. 106, 1994, 51; Int. J. Pharm. 116, 1995, 253; Int. J. Pharm. 84, 1992, R5-R8; J. Phys. Chem. 95, 1991, 989, Langmuir 14, 1998, 3506; Langmuir 11, 1995, 1576; SÖFW 124, 1998, 614-623.

Phospholipid O/W microemulsions containing interface-active pharmaceuticals are described in the literature. Int. J. Pharm. 125,1995, 231; Int. J. Pharm. 89,1993, R9-R12.

Low-viscosity microemulsions for oral applications based on lecithin/ethanol/propylene glycol are described in WO 92/02207. Also described therein is the thickening to give the microemulsion gel using gelatin as water-soluble polymer. A disadvantage of cosmetic applications is the lack of a cosmetic oil phase.

The use of ethanol as amphiphilic cosolvent for the preparation of lecithin-containing microemulsions and also the gelling with polysaccharides such as gelatin or agar is also described in WO 95/31969. Lecithin-containing transparent oil-in-water emulsions thickened with gelatin are also described in FR 2618351. The transparency is achieved by matching the reflective indexes of water and oil phase. Accordingly, no microemulsion is present here.

EP 406162 B1 describes a process for the preparation of a nanoemulsion with triglycerides or fatty acid esters. On p. 2, II. 36-43 and on p. 3 II. 18-28, it is emphasized that the lecithin emulsifier should have a lamellar liquid crystalline structure, which is then processed with a high-pressure homogenizer to give the nanoemulsion. DE 3930928 C2 describes pharmaceutical formulations containing cyclosporin. The microemulsion concentrate used is, in addition to cyclosporin as active ingredient, advantageously propylene glycol or glycofurol as hydrophilic component. On page 6 lines 7 to 12, it is stated that these concentrates represent O/W or W/O macroemulsions. The gel state which is advantageously passed through, which is not to be regarded as a macroemulsion, is not mentioned. In the examples, essentially ethoxylated emulsifiers are used, and lecithin-containing formulations and the procedure in the preparation of the invention forming the basis of this application are not mentioned apart from ex. 1.4. In addition, short-chain ethers such as transcutol and glycofurol are not very suitable for cosmetic purposes due to penetration.

EP 0100448 and DE 3225706 describe phospholipid-containing microemulsions consisting of an ethoxylated glycerol ester, phospholipid and an oil phase. The oil phase used is isopropyl palmitate, glyceryl triacetate or Miglyols. The lipophilic phase is mixed with phospholipid and O/W emulsifier and then diluted with water. A gel state is not passed through here as an intermediate stage. The short-chain alcohol used is ethanol or isopropanol. These ingredients are known as penetration accelerators and are therefore disadvantageous.

EP 760237 describes pharmaceutical preconcentrates which consist of mono-, di-, triglycerides as oil phase, pharmaceutical active ingredient and a phospholipid and one other emulsifier. Dilution of the concentrate in water produces O/W microemulsions. In particular, the formulations prepared in this way should prevent the active ingredient cyclosporin from subsequently precipitating out. A disadvantage is that only coconut oil, castor oil or peanut oil are used as oil phases. An intermediate gel state is not passed through. WO 9709964 describes mixtures of phospholipids and hydrophilic surfactants which, in addition to the oil phase, comprise a “surfactant film modifier”. This is preferably ethanol or a C3-alcohol. On page 7, lines 1-4, it is stated that the mixtures used must be equilibrated for two to 3 days, which may be regarded as a disadvantage. The oil phase used in the examples is only Miglyol 810 (short-chain triglyceride) and isopropyl myristate. The generation of the microemulsion via an intermediate gel state is not disclosed. WO 97/30695 describes microemulsions for intravenous purposes. In this respect, concentrates are firstly prepared which consist of phospholipids, propylene glycol (or PEG), an emulsifier with a high HLB value, an active ingredient and 0-30% of water. Oil components used are triglycerides or else propylene glycol diesters. On page 6 lines 11-13 reference is made to the propylene glycol to be used particularly advantageously. This may also be partially or completely substituted by polyethylene glycol (p. 10, II. 18-19). Alcohols such as ethanol are less suitable for intravenous purposes. On page 23 II. 23-25 and page 24,II. 4-5, it is stated how significant the propylene glycol is for the manufacture of transparent preparations. Formulations without propylene glycol produce milky opaque emulsions following dilution with water. The advantage of a gel state is not recognized. EP 852941 describes nanodispersions which are produced by dissolving the phospholipid in ethanol and subsequently admixing with an unsaturated ethoxylated sorbitan ester and an active ingredient (or oil phase). It is a disadvantage that ethanol has to be used, which may, in particular, lead to increased penetration into the skin and/or can partially or entirely cancel the positive properties of the phospholipids since ethanol has a drying effect. In addition, only triglycerides are solubilized with the process presented above. It is a disadvantage that only sorbitan esters, in particular saturated ones, can be used, meaning that it is necessary to seek very effective antioxidants for product protection of the phospholipids (if unsaturated ones are to be used), which are in any case already to be stabilized. The same disadvantages arise in WO 96/37192, in which sphingo lipids and glycolipids are solubilized. EP 956851 describes nanodispersions which can be prepared in two different ways. The first process relates to the mixing of a membrane-forming molecule (phospholipid), a coemulsifier (ethoxylated) and a lipophilic constituent (oil phase or active ingredient), which are mixed until a homogeneous, clear solution forms (nanodispersion prephase). This prephase is introduced into a water phase without the input of energy (page 2, lines 35-50). On page 2, lines 51-52, it is stated that water is not necessary for mixing the phospholipid/coemulsifier/oil phase mixture. The advantage of the addition of water and the thus induced formation of a gel state (i.e. the advantageous formation of a mesophase) has not been recognized. The second process differs from the first in that the prephase additionally comprises propylene glycol or ethanol. In the examples for the preparation of nanodispersions, only triglycerides are used as typical oil phase, which is disadvantageous. The addition of a lipophilic coemulsifier, which then permits dispensation of ethanol, is not described. DE 3225706 describes liquid active ingredient formulations in the form of concentrates for microemulsions. It is described that, as well as making use of phospholipid, use is made of an OIW coemulsifier with a HLB value of 12-18. The gel state to be passed through according to the invention during the preparation of the microemulsion is not described. The broad variability of the use of different oil phases which arise through the use of a W/O emulsifier or through the use of O/W emulsifiers other than those described, and the advantageous combination of an O/W emulsifier and of a W/O emulsifier in addition to a phospholipid is not described. The disadvantages associated with the use of ethanol for microemulsions, such as skin drying, increased penetration, are not described.

DE 3302898 describes an emulsifying system which comprises a fatty acid or a protein condensate, a polyethoxylated stearyl and a phosphatide. On page 6, line 25 it is stated that emulsoids are understood as meaning emulsions whose particle size is less than one micron. It is known to the person skilled in the art that there are more finely divided emulsions (e.g. PIT emulsions), the particle size of which may be less than one micron. Neither is the gel formation described utilized for forming an O/W microemulsion. WO 9405298 describes “submicron emulsions” for applications around the eye. The reduction of the particle size is achieved by homogenization of a coarsely particulate emulsion at a pressure of a 8000 psi and subsequent filtration (p. 14,II. 18-24). It is a disadvantage that it is not possible here to dispense with high-pressure homogenization. In addition, microemulsions with cationic ingredients are known which, as conditioning agents, facilitate the stylability of hair. A disadvantage here is the use of the cationic additives. Lecithin organogels are described in the literature. Colloid Polymer Science 268, 1990, 356; Colloid J. 58, 1996, 117; Colloid Polym. Sci. 266, 1990, 356; Int. J. Pharm. 137, 1996, 117; J. Phys. Chem. 92, 1988, 829; J. Pharm Sci. 81, 1992, 871; J. Contr. Rel. 34, 1995, 53; Proced. Intem. Symp. Control. Rel. Bioact. Mater. 17, 1990, 421; Progr. Colloid Polym. Sci. 105, 1997, 204; Progr. Colloid Polym. Sci. 106, 1997, 228; Skin. Pharmacol. 9, 1996, 124. These emulsifier-free organogels are obtained by adding small amounts of water to a mixture of organic solvent and lecithin. Here, when water is added, the inverse micelles form cylinder-like water-filled micelles (“wormlike micelles”), which become entangled with one another and thus explain the high viscosity of these mixtures. (Colloid Polym. Sci. 268, 1990, 356).

Strictly speaking, these lecithin gels do not represent microemulsion gels since the dispersed phase is not in droplet form and also a corresponding viscosity-increasing substance for the continuous phase is missing. Neither is it described that these gels of the prior art can also be obtained in the presence of an O/W emulsifier. Neither is it described that these gels can also be obtained in the presence of an O/W emulsifier or a W/O emulsifier. Neither is it described that the gels of the prior art can be converted in the presence of water into other colloidochemical phases, such as, for example, O/W microemulsions and OIW macroemulsions. Neither is it described that, in the presence of an OW emulsifier, gel-like preparations can be obtained which can be converted in a targeted manner into low-viscosity, lecithin-containing OIW microemulsions by dilution with water. It is not described that, in the presence of an OIW emulsifier or a W/O emulsifier, gel-like preparations can be obtained which can be converted in a targeted manner into low-viscosity, lecithin-containing O/W microemulsions by dilution with water.

It was an object of the invention to avoid said disadvantages.

This object is achieved according to the invention.

The invention provides gels or low-viscosity transparent or translucent microemulsions of the oil-in-water type, comprising a water phase and an oil phase, which are essentially composed of low-volatility constituents, comprising: at least one phospholipid and at least one oil-in-water emulsifier and optionally at least one W/O emulsifier, obtainable by adding the water phase with its constituents to the oil phase with its constituents, in particular the phospholipid and the O/W emulsifier and optionally the W/O emulsifier, the phases being mixed with one another and a gel state being achieved, and if a low-viscosity O/W microemulsion is desired, further parts of the water phase are added and the phases are mixed, it being possible, if desired, for the phases to comprise further auxiliaries, additives and/or active ingredients.

The invention also provides a process for the preparation of gels or low-viscosity transparent or translucent microemulsions of the oil-in-water type, comprising a water phase and an oil phase, which is essentially composed of low-volatility constituents, comprising at least one phospholipid and at least one oil-in-water emulsifier and optionally at least one W/O emulsifier, characterized in that a phospholipid is dissolved in the oil phase, optionally with further constituents, and the water phase, optionally with further constituents, is added thereto and the phases are mixed, during which the viscosity increases and, for example, the gels are obtained and, upon the further addition of the water phase, the microemulsions arise, where the oil-in-water emulsifier and optionally the W/O emulsifier can be added to the oil phase or can be added at the gel formation stage or else following preparation of the gels.

The water phase is expediently metered into or added dropwise to the oil phase, e.g. with stirring, until there is an increase in the viscosity or until a gel forms and then the remaining water phase is metered in. The lecithin is advantageously dissolved in the oil phase (optionally at elevated temperature). It is, however, also possible to dissolve the lecithin in the oil at room temperature. The O/W emulsifier and optionally the W/O emulsifier can be added directly to the oil phase or not until the stage of gel formation or following preparation of the lecithin organogel (phospholide/organic solvent). The water phase can be added at room temperature or optionally at elevated temperature.

The components are preferably mixed by stirring, optionally at elevated temperature. In particular, it is thus possible to dispense with an input of energy, e.g. by homogenization.

In the description, by “lecithin” are also meant, for example, the phospholipids, which include, for example, the following substances: phosphatidic acids, the real lecithins, cardiolipins, lysophospholipids, lysolecithins, plasmalogens, phosphosphingolipids, sphingomyelins. Preferred substances are described below.

Phosphatidic acids are glycerol derivatives which have been esterified in the 1-sn- and 2-position with fatty acids (1-sn-position: mostly saturated, 2-position: mostly mono- or polyunsaturated), but on atom 3-sn with phosphoric acid, and are characterized by the general structural formula

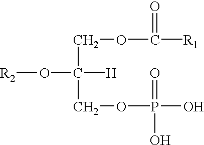

In the phosphatidic acids which occur in human or animal tissue, the phosphate radical is in most cases esterified with amino alcohols such as choline (lecithin=3-sn-phophatidylcholine) or 2-aminoethanol (ethanolamine) or L-serine (cephalin=3-sn-phosphatidylethanolamine or sn-phosphatidyl-L-serine), with myoinositol to give the phosphoinositides [1-(3-sn-phosphatidyl)-D-myoinositols], common in tissues, with glycerol to give phosphatidyl glycerols. Particular preference is given to lecithins (=3-sn-phosphatidylcholine).

Lecithins (or the real lecithins) are characterized, for example, also by the general structural formula

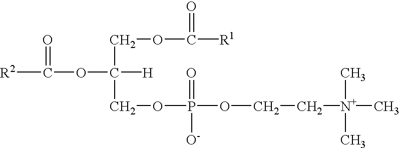

where R¹ and R² are typically unbranched aliphatic radicals having 15 or 17 carbon atoms and up to 4 cis double bonds.

Cardiolipins (1,3-bisphosphatidyl glycerols) are phospholipids of two phosphatidic acids linked via glycerol.

Lysophospholipids are obtained when an acyl radical is cleaved off by a phospholipase A from phospholipids (e.g. lysolecithins).

Lysophospholipids are characterized by the general structural formula

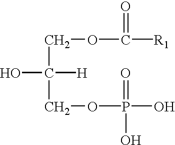

Lysolecithins, for example, are characterized by the general structural formula

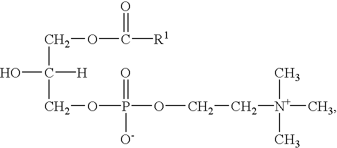

where R and R² are typically unbranched aliphatic radicals having 15 or 17 carbon atoms and up to 4 cis double bonds.

Preferred phospholipids are phosphatidylcholine, phosphatidylethanolamine, phosphatidylinositol or N-acylphosphatidylethanolamine or mixtures of two or more of these compounds.

The phospholipids also include plasmalogens in which an aldehyde (in the form of an enol ether) is bonded in the 1-position instead of a fatty acid; the O-1-sn-alkenyl compounds corresponding to the phosphatidylcholines are called, for example, phosphatidylcholines.

Phosphosphingolipids are based on the basic structure of sphingosine or else phytosphingosine, which are characterized by the following structural formulae:

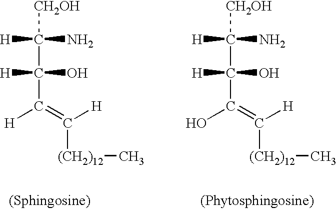

Modifications of sphingolipids are characterized, for example, by the (Sphingosine) (Phytosphingosine) general basic structure

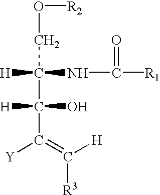

in which R₁ and R₃, independently of one another, are saturated or unsaturated, branched or unbranched alkyl radicals having 1 to 28 carbon atoms, R₂ is chosen from the group: hydrogen atom, saturated or unsaturated, branched or unbranched alkyl radicals having 1 to 28 carbon atoms, sugar radicals, phosphate groups which are unesterified or esterified with organic radicals, sulfate groups which are unesterified or esterified with organic radicals, and Y is either a hydrogen atom, a hydroxyl group or another heterofunctional radical.

Sphingophospholipids:

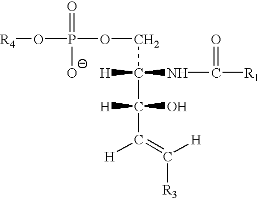

R₁ and R₃ are alkyl radicals, R₄ is an organyl radical.

Sphingomyelins are organophosphorylated sphingolipids of the type

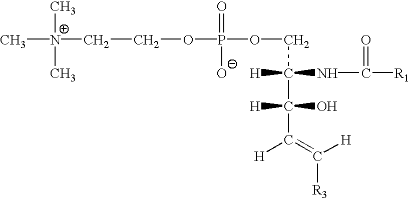

Particularly preferred phospholipids are lecithins. Types of lecithin which are to be used advantageously are chosen from crude lecithins which have been deoiled and/or fractionated and/or spray-dried and/or acetylated and/or hydrolyzed and/or hydrogenated. They are available commercially. Preference is given to soybean lecithins.

Phospholipids which may be used advantageously according to the invention are, for example, available commercially under the trade names Phospholipon 25 (Nattermann), Emulmetik 120 (Lucas Meyer), Stempur E (Stern), Stempur PM (Stern), Nathin 3KE (Stern), Phospholipon 90 (Rhône-Poulenc), Phosopholipon 90 H (Rhône-Poulenc).

In the presence of the OIW emulsifier it is possible for new types of gel to form in which other colloidochemical phases are also present, as in the “pure” lecithin organogels known in the literature such as, for example, lamellar liquid crystals, cubic phases, bicontinuous microemulsion gels, O/W microemulsion gels, inverse hexagonal phases, hexagonal phases, inverse mycellar phases, WO microemulsion gels.

All of these e.g. creamy preparations characterized by an increase in viscosity are referred to here as “gels”. If more water phase is added to the gel, the viscosity decreases and a low-viscosity O/W microemulsion forms. The preferred intermediate gel formation according to the invention (i.e. the corresponding colloidochemical phase) and its targeted breakdown by dilution with water (i.e. the conversion of the colloidochemical phase into another) permits the preparation of finely divided O/W microemulsions. In this way, it is possible for the first time to use a large number of O/W emulsifiers. In addition, the greater variability in the choice of O/W emulsifiers favors a greater variety of cosmetic oil phases. The addition of W/O emulsifiers is advantageous if stability problems arise or if active ingredients are difficult to solubilize. In addition, this makes it possible to dispense with ethanol, meaning that the skin-drying or excessive penetration-promoting disadvantages of the administration system of the prior art are avoided. In this way, it is possible to incorporate a greater number of active ingredients which differ, for example, by virtue of their polarity or their hydrophilicity/lipophilicity more readily into O/W microemulsions. Suitable O/W emulsifiers are described below. Ethoxylated fatty acid esters and fatty acid glycerides, in particular PEG-50 hydrogenated castor oil isostearate PEG-45 palm kernel oil glycerides

polyglycerol esters, in particular Polyglycerol-10 stearate Polyglycerol-10 laurate

ethoxylated glycerol esters, in particular PEG-20 glyceryl laurate PEG-20 glyceryl stearate

fatty acid ethoxylates, in particular PEG-20 monostearate

fatty alcohol ethoxylates, in particular Ceteareth-12 Oleth-15

alkyl ether sulfates, ether carboxylates, in particular Na lauryl ether sulfate

sulfated glycerol esters, in particular Na glyceryl cocoyl sulfate, ammonium glyceryl cocoyl sulfate

acyl lactylates, acyl sarcosinates, acyl glutamates, in particular Na lauroyl lactylate sorbitan esters or derivatives thereof, e.g.

ethoxylated sorbitan esters, in particular PEG-20 sorbitan isostearate PEG-20 sorbitan monooleate can optionally also be used.

The O/W emulsifiers below are preferred.

Advantageously, the polyethoxylated or polypropoxylated or polyethoxylated and polypropoxylated O/W emulsifier or the polyethoxylated or polypropylated or polyethoxylated and polypropoxylated O/W emulsifiers are used which can be chosen from the group

of fatty alcohol ethoxylates of the general formula R—O—(—CH₂—CH₂—O—)ₙ—H, where R is a branched or unbranched alkyl or alkenyl radical and n is a number from 10 to 50

of ethoxylated wool wax alcohols,

of polyethylene glycol ethers of the general formula R—O—(—CH₂—CH₂—O—)ₙ—R′, where R and R′, independently of one another, are branched or unbranched alkyl or alkenyl radicals and n is a number from 10 to 80

of fatty acid ethoxylates of the general formula R—COO—(—CH₂—CH₂—O—)ₙ—H, where R is a branched or unbranched alkyl or alkenyl radical and n is a number from 10 to 40,

of etherified fatty acid ethoxylates of the general formula R—COO—(—CH₂—CH₂—O—)ₙ—R′, where R and R′, independently of one another, are branched or unbranched alkyl or alkenyl radicals and n is a number from 10 to 80,

of esterified fatty acid ethoxylates of the general formula R—COO—(—CH₂—CH₂—O—)ₙ—C(O)—R′, where R and R′, independently of one another, are branched or unbranched alkyl or alkenyl radicals and n is a number from 10 to 80,

of polyethylene glycol glycerol fatty acid esters of saturated and/or unsaturated, branched and/or unbranched fatty acids and [lacuna] a degree of ethoxylation between 3 and 50,

of ethoxylated sorbitan esters having a degree of ethoxylation of from 3 to 100

of cholesterol ethoxylates having a degree of ethoxylation between 3 and 50,

of ethoxylated triglycerides having a degree of ethoxylation between 3 and 150,

of alkyl ether carboxylic acids of the general formula R—O—(—CH₂—CH₂—O—)ₙ—CH₂—COOH or cosmetically or pharmaceutically acceptable salts thereof, where R is a branched or unbranched alkyl or alkenyl radical having 5-30 carbon atoms and n is a number from 10to30,

of polyoxyethylene sorbitol fatty acid esters based on branched or unbranched alkanoic or alkenoic acids and having a degree of ethoxylation of from 5 to 100, for example of the Sorbeth type,

of alkyl ether sulfates or the acids on which these sulfates are based, of the general formula R—O—(—CH₂—CH₂—O—)ₙ—SO₃—H with cosmetically or pharmaceutically acceptable cations, where R is a branched or unbranched alkyl or alkenyl radical having 5-30 carbon atoms and n isg a number from 1 to 50,

of fatty alcohol propoxylates of the general formula R—O—(—CH₂—CH(CH₃)—O—)ₙ—H, where R is a branched or unbranched alkyl or alkenyl radical and n is a number from 10 to 80,

of polypropylene glycol ethers of the general formula R—O—(—CH₂—CH(CH₃)—O—)ₙ—R′, where R and R′, independently of one another, are branched or unbranched alkyl or alkenyl radicals and n is a number from 10 to 80,

of propoxylated wool wax alcohols,

of etherified fatty acid propoxylates of the general formula R—COO—(—CH₂—CH(CH₃)—O—)ₙ—R′, where R and R′ independently of one another, are branched or unbranched alkyl or alkenyl radicals and n is a number from 10 to 80,

of esterified fatty acid propoxylates of the general formula

R—COO—(—CH₂—CH(CH₃)—O—)ₙ—C(O)—R′, where R and R′ independently of one another, are branched or unbranched alkyl or alkenyl radicals and n is a number from 10 to 80,

of fatty acid propoxylates of the general formula R—COO—(—CH₂—CH(CH₃)—O—)ₙ—H, where R is a branched or unbranched alkyl or alkenyl radical and n is a number from 10 to 80,

of polypropylene glycol glycerol fatty acid esters of saturated and/or unsaturated, branched and/or unbranched fatty acids and [lacuna] a degree of propoxylation between 3 and 80

of propoxylated sorbitan esters having a degree of propoxylation of from 3 to 100

of cholesterolpropoxylates having a degree of propoxylation of from 3 to 100

of propoxylated triglycerides having a degree of propoxylation of from3to100

of alkyl ether carboxylic acids of the general formula R—O—(—CH₂—CH(CH₃)—O—)ₙ—CH₂—COOH, or cosmetically or pharmaceutically acceptable salts thereof, where R is a branched or unbranched alkyl or alkenyl radical and n is a number from 3 to 50,

of alkyl ether sulfates or the acids on which, the sulfates are based of the general formula R—O—(—CH₂—CH(CH₃)—O—)ₙ—SO₃—H with cosmetically or pharmaceutically acceptable cations, where R is a branched or unbranched alkyl or alkenyl radical having 5-30 carbon atoms and n is a number from 1 to 50,

of fatty alcohol ethoxylates/propoxylates of the general formula R—O—Xₙ—Yₘ—H, where R is a branched or unbranched alkyl or alkenyl radical, where X and Y are not identical and in each case are either an oxyethylene group or an oxypropylene group and n and m independently of one another are numbers from 5 to 50,

of polypropylene glycol ethers of the general formula. R—O—Xₙ—Yₘ—R′, where R and R′, independently of one another, are branched or unbranched alkyl or alkenyl radicals, where X and Y are not identical and in each case are either an oxyethylene group or an oxypropylene group and n and m, independently of one another are numbers from 5 to 100,

of etherified fatty acid propoxylates of the general formula R—COO—Xₙ—Yₘ—R′, where R and R′, independently of one another, are branched or unbranched alkyl or alkenyl radicals, where X and Y are not identical and in each case are either an oxyethylene group or an oxypropylene group and n and m, independently of one another, are numbers from 5 to 100,

of fatty acid ethoxylates/propoxylates of the general formula R′COO′Xₙ—Yₘ—H, where R is a branched or unbranched alkyl or alkenyl radical, where X and Y are not identical and in each case are either an oxyethylene group or an oxypropylene group and n and m, independently of one another, are numbers from 5 to 50.

Diacetyltartaric esters of mono/diglycerides

of partially neutralized esters of monoglycerides and/or diglycerides of saturated fatty acids with alpha-hydroxy acids.

Suitable are glycerol esters of α-hydroxycarboxylic acids and saturated fatty acids chosen from the group of compounds represented by the generic formula

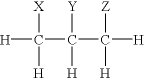

where X, Y and Z, independently of one another, are chosen from the group

(1) OH,

(2) from the group of saturated branched and unbranched carboxylic acid radicals according to the formula

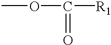

where R is a branched or unbranched alkyl radical having 10-24 carbon atoms and

(3) from the group of α-hydroxycarboxylic acid radicals according to the formula

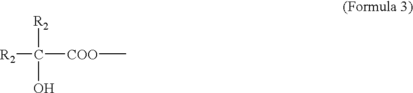

(a) where R₂ and R₃, independently of one another, are chosen from the group

(a1) H,

(a2) branched or unbranched C₁₋₂₅-alkyl,

(a3) branched or unbranched C₁₋₂₅-alkyl substituted by one or more carboxyl groups and/or hydroxyl groups and/or aldehyde groups and/or oxo groups (keto groups) or

(b) where the a-carbon atom of the a-hydroxycarboxylic acid together with R₂ and R₃ forms an

(b1) unsubstituted cycloalkyl group having 3 to 7 ring atoms or a

(b2) cycloalkyl group having 3 to 7 ring atoms and substituted by one or more carboxyl groups and/or hydroxyl groups and/or oxo groups (keto groups) and/or branched and/or unbranched C₁₋₂₅-alkyl groups, and where, of the radicals X, Y and Z, only one must be a radical according to formula 3, only one must be a radical according to formula 2 and only one must be an OH group.

The glycerol esters of a-hydroxycarboxylic acids and saturated fatty acids for the purposes of the present invention are particularly advantageously chosen from the group in which the a-hydroxycarboxylic acid radical is a lactic acid radical

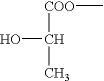

or a citric acid radical

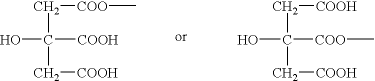

It is also advantageous to choose the fatty acid radical such that R₁ is a C₁₃-C₁₉-alkyl radical.

Such lactic acid esters are available, for example, under the product name “LACTODAN B30” from Grinsted Prods.

Such citric acid esters are available, for example, under the product name “IMWITOR 370” from the company Hüls AG.

water-dispersible W/O emulsifiers

acyl lactylates of the formula R—C(O)O—CH(CH₃)—C(O)O—CH(CH₃) CO₂⁻M⁺, where R is a saturated and/or unsaturated, branched and/or unbranched fatty acid having 6 to 26 carbon atoms.

acyl glutamates of the formula R—C(O)NHCH(COO⁻, M⁺)CH₂CH₂COO⁻M⁺, where R is a saturated and/or unsaturated, branched and/or unbranched fatty acid having 6 to 26 carbon atoms.

acyl sarcosinates of the formula R—C(O)—N(CH₃)CH₂COO⁻M⁺, where R is a saturated and/or unsaturated, branched and/or unbranched fatty acid having 6 to 26 carbon atoms.

isethionates of the formula RC(O)—O—CH₂CH₂—SO₃⁻M⁺, where R is a saturated and/or unsaturated, branched and/or unbranched fatty acid having 6 to 26 carbon atoms.

sulfosuccinates of the formula

M⁺,—O—C(O)—CH₂—CH(SO₃—M+)—C(O)—O—R, where R is a saturated and/or unsaturated, branched and/or unbranched fatty acid having 6 to 26 carbon atoms.

alaninates of the formula CH₃CH₂N(CH₃)(C₁₂H₂₅)C(O)O⁻M⁺

amphoacetates of the formula R—C(O)—NH—CH₂CH₂—N(CH₂CH₂OH)—CH₂COO⁻, M⁺

alkyl glycosides, alkyl polyglycosides,

esters of hydroxy acids

Particular preference is given to: PEG-50 hydrogenated castor oil isostearate, PEG-45 palm kernel oil glycerides, polyglycerol-10 stearate, polyglycerol-10 laurate, PEG-20 glyceryl laurate, PEG-20 glyceryl stearate, PEG-20 monostearate, ceteareth-12, oleth-15, Na lauryl ether sulfate, sodium glyceryl cocoyl sulfates, sodium lauroyl lactylate, sodium cocoyl glutamates, sodium Icocoyl sarcosinates, PEG-20 sorbitan isostearate, PEG-20 sorbitan monooleate, diacetyl tartaric acid mono/dilinoleates, glyceryl linoleate citrate, sodium laureth-11 carboxylate, polyethylene glycol(30) cholesteryl ether, polyethylene glycol(60) evening primrose glycerides, lauryl glycoside, C12-C13-alkyl malic acid esters, C12-C13 tartaric acid esters.

It is, however, also possible not to use any sorbitan esters or sorbitan ester derivatives in the preparations according to the invention.

The oils and fats customary in cosmetics can be used as oil phase.

The process according to the invention permits the preparation of finely divided microemulsions (the droplet size is about 10-100 nm) with a large number of typical oil phases: e.g. ethers (dicarprylyl ether), triglycerides (caprylic capric triglycerides), alcohols (octyldodecanol), ester oils (cetearyl isononanoate), hydrocarbons (dioctyl cyclohexane), paraffins, silicone oils (cyclomethicone) and mixtures of these oil phases.

Where appropriate, the oil phase of the preparations in particular may also comprise sphingolipids and/or glycolipids of synthetic or natural origin, in particular ceramides, sphingomyelins, cerebrosides and/or gangliosides.

The proportion of these lipids may be e.g. 0 to 10% by weight, preferably 0-2% by weight, in particular 0-1% by weight, in each case based on the total weight of the preparations.

In addition, the process presented above opens up the possibility of also utilizing the viscosity-increased states described previously, such as, for example, gels, as administration system.

Thus, these gels can be applied by the consumer, for example as hairgel. Dilution of these gels then leads, depending on the O/W emulsifier used and oil phase used, to O/W microemulsions or O/W macroemulsions on the scalp. In addition, shower oils (foaming, nonfoaming) can be applied topically with utilization of the gel phases according to the invention. The shower water converts the gel on the skin into a water-continuous microemulsion or macroemulsion. In this case, the phospholipid added and further ingredients in the preparation remain on the skin (refatting). In addition, these gels can advantageously be used for removing skin impurities. The gels have the advantageous property of solubilizing lipid-soluble impurities of the skin. These face/body cleansing gels can then be diluted with water by the user, the sebum being solubilized in the oil droplets, thus enabling pore-deep cleansing of the skin. At the same time, some of the phospholipid remains on the skin and thus increases the moisture content.

For the gels, preference is given to the following percentage amounts by weight, in each case based on the total weight of the preparations:

| | |
|---|---|
| Lecithin: | 0.1-50% |
| O/W emulsifier: | 0.1-70% |
| Oil phase: | 5-90% |
| Additives for the | 0.01-15% |
| oil phase: | |
| Additives for the | 0.01-35% |
| water phase: | |
| Water | 0.1-75% water |
| Lecithin: | 0.1-40% |
| O/W emulsifier: | 0.1-70% |
| W/O emulsifier | 01.-50% |
| Oil phase: | 5-90% |
| Additives for the | 0.01-15% |
| oil phase: | |
| Additives for the | 0.01-35% |
| water phase: | |
| Water | 0.1-75% water |

For the microemulsion according to the invention, preference is given to the following percentage amounts by weight, in each case based on the total weight of the preparations:

| Lecithin: | 0.01-10%, in particular 0.1-5.0% |
|---|---|
| O/W emulsifier: | 0.01-60%, in particular 0.1-10% |
| Oil phase: | 0.01-50%, in particular 0.1-30% |
| Additives for the oil phase: | 0.01-20%, in particular 0.1-15% |
| Additives for the water phase: | 0.01-80%, in particular 0.1-60% |
| Water | 40-99% |
| Lecithin: | 0.01-10%, in particular 0.1-5.0% |
| O/W emulsifier: | 0.01-60%, in particular 0.1-10% |
| W/O emulsifier | 0.01-10%, in particular 0.1-5.0% |
| Oil phase: | 0.01-50%, in particular 0.1-30% |
| Additives for the oil phase: | 0.01-20%, in particular 0.1-15% |
| Additives for the water phase: | 0.01 -80%, in particular 0.1-60% |
| Water | 40-99% |

Additives may also be auxiliaries or active ingredients.

The lecithin/O/W emulsifier weight ratio in the preparations according to the invention can vary, e.g. from 1:30 to 2:1. The lecithin/OW emulsifier ratio is preferably 1:15 to 1:1. The lecithin/OW emulsifier ratio is particularly preferably 1:6 to 1:1.5.

The gels and microemulsions according to the invention can optionally also comprise one or more water-in-oil emulsifiers. The W/O emulsifiers are to be used particularly advantageously if, for example, active ingredients are to be solubilized in the microemulsions which tend toward storage instabilities only in the presence of the O/W emulsifier and of the lecithin. Preference is given to emulsifiers with a HLB value in the range from 1-10. Preference is given to using the following W/O emulsifiers:

one or more polyethoxylated W/O emulsifiers and/or

one or more polyproxylated W/O emulsifiers and/or

one or more polyethoxylated and polypropoxylated W/O emulsifiers and/or

one or more monoesters, diesters, polyesters, polyols as W/O emulsifiers and/or

one or more monoethers of polyols and esters thereof as W/O emulsifiers and/or

one or more sorbitan esters as W/O emulsifiers and/or

one or more silicone emulsifiers as W/O emulsifiers and/or

one or more fatty alcohols or fatty acids as W/O emulsifiers and/or

one or more methylglucose esters as W/O emulsifiers,

where this W/O emulsifier is chosen from the group o

fatty alcohol ethoxylates of the general formula R—O—(—CH₂—CH₂—O)ₙ—H, where R is a branched or unbranched alkyl, aryl or alkenyl radical and n is a number from 1 to 10

polyethylene glycol ethers of the general formula R—O—(—CH₂—CH₂—O—)ₙ—R′, where R and R′, independently of one another, are branched or unbranched alkyl or alkenyl radicals and n is a number from 1 to 30

fatty acid ethoxylates of the general formula R—COO—(—CH₂—CH₂—O—)ₙ—H, where R is a branched or unbranched alkyl or alkenyl radical and n is a number from 1 to 20,

esterified fatty acid ethoxylates of the general formula R—COO—(—CH₂—CH₂—O—)ₙ—C(O)—R′, where R and R′, independently of one another, are branched or unbranched alkyl or alkenyl radicals and n is a number from 1 to 20,

esterified fatty acid ethoxylates of the general formula R—COO—(—CH₂—CH₂—O—)ₙ—C(O)—R′, where R and R′, independently of one another, are branched or unbranched alkyl, hydroxyalkyl or alkenyl radicals and n is a number from 1 to 40,

etherified fatty acid ethoxylates of the general formula

R—COO—(—CH₂—CH₂—O—)ₙ—R′, where R and R′, independently of one another, are branched or unbranched alkyl or alkenyl radicals and n is a number from 1 to 40

fatty alcohol propoxylates of the general formula R—O—(—CH₂—CH(CH₃)—O—)ₙ—H, where R is a branched or unbranched alkyl or alkenyl radical and n is a number from 1 to 30,

polyoxyethylene sorbitan fatty acid esters based on branched or unbranched alkanoic or alkenoic acids and having a degree of ethoxylation of from 1 to 10

cholesterol ethoxylates having a degree of ethoxylation between 1 and 10,

ethoxylated glycerides having a degree of ethoxylation of from 1 to 30

ethoxylated triglycerides having a degree of ethoxylation between 1 and 30,

monoglycerol ethers of the type R—O—CH₂—C(H)OH—CH₂OH where R are a branched or unbranched alkyl, aryl or alkenyl radical and

monoglycerol esters of the type RC(O)OCH₂—C(H)OH—CH₂OH where R are a branched or unbranched alkyl, hydroxyalkyl, aryl or alkenyl radical

diglycerol esters of the type RC(O)OCH₂—C(H)OH—CH₂OC(O)R′where where R and R′, independently of one another, are branched or unbranched alkyl, hydroxyalkyl, or alkenyl radicals and n is a number from 1 to 30 or,

polyglycerol monoesters or diesters or polyesters, where the fatty acids, independently of one another, are branched or unbranched alkyl, hydroxyalkyl or alkenyl radicals,

pentaerythritol esters, where the fatty acids, independently of one another, are branched or unbranched alkyl, hydroxyalkyl or alkenyl radicals,

propylene glycol esters, where the fatty acids, independently of one another, are branched or unbranched alkyl, hydroxyalkyl or alkenyl radicals,

sorbitan esters, where the fatty acids, independently of one another, are branched or unbranched alkyl, hydroxyalkyl or alkenyl radicals,

fatty alcohols R—OH and fatty acids RCOOH, where R are a branched or unbranched alkyl or alkenyl radical,

silicone emulsifiers

methylglucose esters, where the fatty acids, independently of one another, are branched or unbranched alkyl, hydroxyalkyl or alkenyl radicals

fatty alcohols having 8 to 30 carbon atoms,

diglycerol ethers, where the fatty acids, independently of one another, are branched or unbranched alkyl hydroxyalkyl or alkenyl radicals

sucrose esters, where the fatty acids, independently of one another, are branched or unbranched alkyl, hydroxyalkyl or alkenyl radicals

Particularly advantageous W/O emulsifiers are glyceryl monoisostearate, glyceryl monoisostearate, diglyceryl monostearate, diglyceryl monoisostearate, propylene glycol monostearate, sorbitan monoisostearate, cetyl alcohol, archidyl alcohol, selachyl alcohol, chimyl alcohol, glyceryl monolaurate, glyceryl monocaprate, glyceryl monocaprylate, steareth-2, polyethylene glycol(4) stearate, polyethylene glycol(12) dilaurate, polyethylene glycol(8) distearate. Polyethylene glycol(20) sorbitan trioleate, polyethylene glycol(20) sorbitan triisostearate, polyethylene glycol(5) phytosterol, polyethylene glycol(20), glyceryl tristearate, polyethylene glycol(5) glyceryl stearate, 2-ethylhexyl glycerol ether, polyglyceryl-2 dipolyhydroxystearate, cetyldimethicone copolyol, isostearic acid, linoleic acid, linolenic acid.

The lecithin/(OW emulsifier and W/O emulsifier) weight ratio in the preparations according to the invention can vary, e.g. from 1:30 to 2:1. The lecithin/(O/W emulsifier and W/O emulsifier) ratio is preferably 1:15 to 1:1.

The lecithin/O/W emulsifier and W/O emulsifier is particularly preferably 1:6 to 1:1.5.

The gels or microemulsions according to the invention can have high oil phase fractions. In particular, they can be used for the treatment of skin roughness and for skin smoothing and they bring about an increase in skin moisture.

The preparations described below can be gels or microemulsions according to the invention.

The gels or microemulsions according to the invention can be used as administration systems for e.g. cosmetic or e.g. dermatological active ingredients, additives, or auxiliaries. They are preferably applied topically.

The gels according to the invention can be used as administration system (make-up remover, hair gel, face cleansing gel, bodycare gel). The gels according to the invention can be converted in the presence of water into other colloidochemical phases, such as, for example, O/W microemulsions and O/W macroemulsions. In the presence of an O/W emulsifier and optionally a W/O emulsifier, gel-like preparations can be obtained which can be converted into low-viscosity, lecithin-containing O/W microemulsions in a targeted manner by dilution with water.

If the gels and microemulsions according to the invention are bases for cosmetic deodorants/antiperspirants, then all customary active ingredients may advantageously be used, for example odor maskers, such as the customary perfume constituents, odor absorbers, for example the phyllosilicates described in patent laid-open specification DE-P 40 09 347, and of these in particular montmorillonite, kaolinite, ilite, beidelite, nontronite, saponite, hectorite, bentonite, smectite, and also, for example, zinc salts of ricinoleic acid. Germicidal agents are likewise suitable to be incorporated into the microemulsions according to the invention.

Advantageous substances are, for example, 2, 4, 4′-trichloro-2′-hydroxydiphenyl ether (Irgasan), 1,6-di(4-chlorophenylbiguanido)hexane (chlorhexidine), 3, 4, 4′-trichlorocarbanilide, quaternary ammonium compounds, oil of cloves, mint oil, oil of thyme, triethyl citrate, farnesol (3, 7, 11-trimethyl-2, 6, 10-dodecatrien-1-ol) and the active agents described in patent laid-open specifications DE-37 40 186, DE-39 38 140, DE-42 04 321, DE-42 29 707, DE-42 29 737, DE-42 37 081, DE-43 09 372, DE-43 24 219.

The customary antiperspirant active ingredients can likewise advantageously be used in the microemulsions according to the invention, in particular astringents, for example basic aluminum chlorides.

The amount of deodorant active ingredients and/or antiperspirant active ingredients can, for example, be 0.001 to 50% by weight, preferably 0.1 to 35% by weight, in each case based on the total weight of the preparation.

The cosmetic deodorants according to the invention can be in the form of aerosols, that is to say preparations which can be sprayed from aerosol containers, squeezable bottles or by a pump device, or in the form of liquid compositions which can be applied by means of roll-on devices, but also in the form of microemulsions which can be applied from normal bottles and containers.

Suitable propellants for cosmetic deodorants according to the invention which can be sprayed from aerosol containers are the customary known readily volatile, liquefied propellants, for example hydrocarbons (propane, butane, isobutane), which can be used on their own or as a mixture with one another. Compressed air can also be used advantageously.

The person skilled in the art naturally knows that there are propellant gases which are nontoxic per se and would in principle be suitable for the present invention, but which should nevertheless be omitted because of an unacceptable impact on the environment or other concomitant circumstances, in particular chlorofluorocarbons (CFCs).

Moreover, it has surprisingly been found that if propellants which are soluble in the oil phase are used, i.e. for example customary propane/butane mixtures, the O/W microemulsions according to the invention are sprayed not simply as aerosol droplets, but develop to give fine-bubbled, rich foams as soon as those systems charged with such propellants experience decompression.

Such after-foaming preparations are therefore likewise to be regarded as advantageous embodiments of the present invention with an independent inventive step.

If propellants which are insoluble in the oil phase are used, the O/W microemulsions according to the invention are sprayed as aerosol droplets.

Also favorable are those cosmetic and dermatological preparations which are in the form of a sunscreen. In addition to the active ingredient combinations according to the invention, these preferably additionally comprise at least one UVA filter substance and/or at least one UVB filter substance and/or at least one inorganic pigment.

It is, however, also advantageous for the purposes of the present inventions to provide cosmetic and dermatological preparations whose main purpose is not protection from sunlight, but which nevertheless comprise a content of UV protection substances. Thus, for example, UV-A or UV-B filter substances are usually incorporated into day creams.

Preparations according to the invention may advantageously comprise substances which absorb UV radiation in the UVB region, the total amount of filter substances being, for example, 0.1% by weight to 30% by weight, preferably 0.5 to 10% by weight, in particular 1 to 6% by weight, based on the total weight of the preparations.

The UV filters may be oil-soluble or water-soluble. Examples of oil-soluble substances are:

3-benzylidenecamphor and its derivatives, e.g. 3-(4-methylbenzylidene)camphor,

4-aminobenzoic acid derivatives, preferably 2-ethylhexyl 4-(dimethylamino)benzoate and amyl 4-(dimethylamino)benzoate;

esters of cinnamic acid, preferably 2-ethylhexyl 4-methoxycinnamate, isopentyl 4-methoxycinnamate;

esters of salicylic acid, preferably 2-ethylhexyl salicylate, 4-isopropylbenzyl salicylate and homomethyl salicylate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably di(2-ethylhexyl) 4-methoxybenzalmalonate;

2, 4, 6-trianilino(p-carbo-2′-ethyl-1′-hexyloxy)-1, 3, 5-triazine

Advantageous water-soluble substances are:

2-phenylbenzimidazol-5-sulfonic acid and salts thereof, e.g. sodium, potassium or triethanolammonium salts,

sulfonic acid derivatives of benzophenones, preferably 2-hydroxy-4-methoxybenzophenone-5-sulfonic acid and its salts;

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

The list of said UVB filters which can be used according to the invention is not of course intended to be limiting.

The invention also provides the combination of a UVA filter with a UVB filter or a cosmetic or dermatological preparation according to the invention which also comprises a UVB filter.

It may also be advantageous to use in preparations according to the invention UVA filters which are customarily present in cosmetic and/or dermatological preparations. Such substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. Preparations which comprise these combinations are also provided by the invention. The amounts of UVA filter substances used are the same as those given for UVB filter substances.

Cosmetic and/or dermatological preparations according to the invention can also comprise inorganic pigments which are customarily used in cosmetics for protecting the skin against UV rays. These are oxides of titanium, zinc, iron, zirconium, silicon, manganese, aluminum, cerium and mixtures thereof, and also modification in which the oxides are the active agents. Particular preference is given to pigments based on titanium dioxide. The amounts used may be those given for the above combinations.

A surprising property of the present invention is that preparations according to the invention are very good vehicles for cosmetic or dermatological active ingredients into the skin, advantageous active ingredients being antioxidants which are able to protect the skin against oxidative stress.

According to the invention, the preparations advantageously comprise one or more antioxidants. Favorable, but nevertheless optional, antioxidants are all antioxidants customary or suitable for cosmetic and/or dermatological applications. It is advantageous here to use antioxidants as the sole class of active ingredient, say, when a cosmetic or dermatological application is a priority, such as the combating of oxidative stress of the skin. It is, however, also favorable to provide the microemulsions according to the invention with a content of one or more antioxidants if the preparations are to serve another purpose, e.g. as deodorants or sunscreen compositions.

The antioxidants are particularly advantageously chosen from the group consisting of amino acids (e.g. histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, gamma-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine-sulfoximines, homocysteine-sulfoximine, buthionine-sulfones and penta-, hexa- and heptathionine-sulfoximine) in very low tolerated doses (e.g. pmol to μmol/kg), and also (metal) chelating agents (e.g. α-hydroxy fatty acids, α-hydroxypalmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol derivatives thereof, vitamin C and derivatives (e.g. ascorbyl palmitates, Mg ascorbyl phosphates, and ascorbyl acetates), tocopherols and derivatives (e.g. vitamin E acetate) vitamin A and derivatives (vitamin A palmitate), and coniferyl benzoate of benzoin resin, rutinic acid and derivatives thereof, ferulic acid and derivatives thereof, butyl hydroxytoluene, butyl hydroxyanisol, nordihydroguaiacic acid, nordihydroguaiaretic acid, trihydroxybutyrophenone, uric acid and derivatives thereof, zinc and derivatives thereof (e.g. ZnO, ZnSO₄) selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

Oil-soluble antioxidants may be used particularly advantageously for the purposes of the present invention.

The amount of antioxidants (one or more compounds) in the preparations is preferably 0.001 to 30% by weight, particularly preferably 0.05-20% by weight, in particular 1-10% by weight, based on the total weight of the preparation.

If vitamin E and/or derivatives thereof are the antioxidant(s), it is advantageous to choose their respective concentrations from the range from 0.001-10% by weight, based on the total weight of the formulation.

If vitamin A or vitamin A derivatives, or carotenes or derivatives thereof are the antioxidant(s), it is advantageous to choose their respective concentrations from the range from 0.001-10% by weight, based on the total weight of the formulation.

The person skilled in the art is of course aware that exacting cosmetic preparations are usually inconceivable without the customary auxiliaries and additives. These include, for example, bodying agents, fillers, perfume, dyes, emulsifiers, additional active ingredients such as vitamins or proteins, light protection agents, stabilizers, insect repellents, alcohol, water, salts, antimicrobial, proteolytic or keratolytic substances etc.

If desired, the water phase of the O/W microemulsions according to the invention can also comprise thickeners, so that the overall preparation appears gel-like and is to be regarded as a microemulsion gel. Suitable thickeners have proven to be, for example, carrageenan or PEG-4 rapeseed amides and laureth-2 amide MEA.

According to the invention, active ingredients can also very advantageously be chosen from the group of lipophilic active ingredients, in particular from the following group:

acetylsalicylic acid, atropine, azulene, hydrocortisone and derivatives thereof e.g. hydrocortisone-17 valerate, vitamins, e.g. ascorbic acid and derivatives thereof, vitamins of the B and D series, very preferably vitamin B₁, vitamin B₁₂ and vitamin D₁, but also bisabolol, unsaturated fatty acids, namely the essential fatty acids (also often called vitamin F), in particular gamma-linolenic acid, oleic acid, eicosapentaenoic acid, docosahexaenoic acid and derivatives thereof, chloramphenicol, caffeine, prosaglandins, thymol, camphor, extracts or other products of plant and animal origin, for example evening primrose oil, borage oil or currant kernel oil, fish oils, cod-liver oil and also ceramides and ceramide-like compounds.

Although the use of hydrophilic active ingredients is of course also favored according to the invention, a further advantage of the microemulsions according to the invention is that the high number of very finely divided droplets makes precisely oil-soluble or lipophilic active ingredients bioavailable with particularly high effectiveness.

It is also advantageous to choose the active ingredients from the group of refatting substances, for example purcellin oil, Eucerite® and Neocerite®.

It is also possible and, in some instances, advantageous to add washing-active surfactants to the preparations according to the invention. Aqueous cosmetic cleansing agents according to the invention or low-water or anhydrous cleansing agent concentrates intended for aqueous cleansing may comprise cationic, anionic, nonionic and/or amphoteric surfactants, for example conventional soaps, e.g. fatty acid salts of sodium, alkyl sulfates, alkyl ether sulfates, alkane- and alkylbenzenesulfonates, sulfoacetates, sulfobetaines, sarcosinates, amidosulfobetaines, sulfosuccinates, sulfosuccinic monoesters, alkyl ethyl carboxylates, protein fatty acid condensates, alkylbetaines and amidobetaines, fatty acid alkanolamides, polyglycol ether derivatives.

Cosmetic preparations which are cosmetic cleansing preparations for the skin may be in liquid or semisolid form, for example as gels or microemulsions. They preferably comprise at least one anionic, cationic, nonionic or amphoteric surface-active substance or mixtures thereof, optionally electrolytes and auxiliaries, as are customarily used for this purpose. The surface-active substance can preferably be present in a concentration between 1 and 30% by weight in the cleansing preparations, based on the total weight of the preparations.

Cosmetic preparations which are shampoos preferably comprise at least one anionic, nonionic or amphoteric surface-active substance or mixtures thereof, optionally electrolytes and auxiliaries, as are customarily used for this purpose. The surface-active substance can preferably be present in a concentration between 1 and 50% by weight in the cleansing preparations, based on the total weight of the preparations. Cetyltrimethylammonium salts, for example, are to be used advantageously.

The preparations intended for cleansing the hair or the skin comprise, apart from the abovementioned surfactants, water and optionally the additives customary in cosmetics, for example perfume, thickeners, dyes, deodorants, antimicrobial substances, refatting agents, complexing and sequestering agents, pearly luster agents, plant extracts, vitamins, active ingredients and the like.

Despite their oil content, the preparations according to the invention surprisingly have very good foam development, high cleansing power and have a high regeneration action with regard to the general condition of the skin. In particular, the preparations according to the invention have a skin-smoothing action, reduce the feeling of dryness of the skin and make the skin supple.

According to the invention, it is, for example, possible to apply a mixture of lecithin/PEG-20 sorbitan isostearate/octyidodecanol/glycerol to the hair such that, for example, a stay-in conditioner product arises. In addition, the products can also be provided with propellant gas and be applied into the hair (or onto the skin) as a mousse. According to the invention, it is also possible to topically apply a mixture of lecithin/PEG-20 sorbitan isostearate/carprylic/capric triglyceride/glycerol and to achieve a significant moisturization of the skin, skin smoothing and a reduction in skin flakiness. In particular, it is also advantageous to use octyidodecanol or cetearyl isonanoate or diocyticyclohexane as oil phase instead of the triglyceride and to achieve the advantages described for the triglycerides.

If the gels or microemulsions according to the invention are to be used for haircare, they may comprise the customary constituents, usually, for example, film-forming polymers. Of such polymers having at least partially quaternized nitrogen groups (called “film formers” below), those which are preferably suitable are those chosen from the group of substances which carry the name, according to INCI nomenclature (International Nomenclature Cosmetic Ingredient) “polyquaternium”, for example:

| | |
|---|---|
| Polyquaternium-2 | (chemical abstract No. 63451-27-4, e.g. Mirapol ® A- |
| | 15) |
| Polyquaternium-5 | (copolymer of acrylamide and β-methacryloxyethyltri- |
| | methylammonium methosulfate, CAS No. 26006-22-4) |
| Polyquaternium-6 | (homopolymer of N,N-dimethyl-N-2-propenyl-2- |
| | propene-1-aminium chloride, CAS No. 26062-79-3, |
| | e.g. Merquat ® 100 |
| Polyquaternium-7 | N,N-dimethyl-N-2-propenyl-2-propene-1- |
| | aminiumchloride, polymer with 2-propeneamide, CAS |
| | No. 26590-05-6, e.g. Merquat ® S |
| Polyquaternium-10 | quaterrnary ammonium salt of hydroxyethylcellulose, |
| | CAS No. 53568-66-4, 55353-19-0, 54351-50-7, |
| | 68610-92-4, 81859-24-7 e.g.Celquat ® SG-230M, |
| Polyquaternium-11 | vinylpyrrolidone/dimethylaminoethyl methacrylate |
| | copolymer/diethyl sulfate reaction product, CAS No. |
| | 53633-54-8, e.g. Gafquat ® 755N |
| Polyquaternium-16 | vinylpyrrolidone/vinylimidazolinium methochloride |
| | copolymer, CAS No. 29297-55-0, e.g. Luviquat ® HM |
| | 552 |
| Polyquaternium-17 | CAS No. 90624-75-2, e.g. Mirapolp ® AD-1 |
| Polyquaternium-19 | quaternized water-soluble polyvinyl alcohol |
| Polyquaternium-20 | quaternized polyvinyl octadecyl ether dispersible in |
| | water |
| Polyquaternium-21 | polysiloxane-polydimethyl-dimethylammonium acetate |
| | copolymer, e.g. Abil ® B 9905 |
| Polyquaternium-22 | dimethyldiallylammonium chloride/acrylic acid |
| | copolymer, CAS No. 53694-7-0, e.g. Merquat ® 280 |
| Polyquaternium-24 | polymeric quaternary ammonium salt of |
| | hydroxyethylcellulose, reaction product with an |
| | epoxide substituted by lauryldimethylammoniuim, CAS |
| | No. 107987-23-5, e.g. Quatrisoft ® LM-200 |
| Polyquaternium-28 | vinylpyrrolidone/methacrylanlidopropyltrimethyl |
| | ammonium chloride copolymer, e.g. Gafquat ® HS-100 |
| Polyquaternium-29 | e.g. Lexquat ® CH |
| Polyquaternium-31 | CAS No. 136505-02-7, e.g. Hypan ® QT 100 |
| Polyquaternium-32 | N,N,N-trimethyl-2-[(2-methyl-1-oxo-2-propenyl)oxy] |
| | ethaneaminium chloride, polymer with 2-propenamide, |
| | CAS No. 35429-19-7 |
| Polyquaternium-37 | CAS No. 26161-33-1 |

Cetryltrimethylammonium salts, such as CTAB, CTAC. Preparations for haircare according to the invention advantageously comprise 0.01-5% by weight of one or more film formers, preferably 0.1-3% by weight, in particular 0.2-2% by weight, in each case based on the total weight of the preparations. Such embodiments of the preparations according to the invention care for hair which has been damaged or worn out by environmental influences, or protect against such environmental influences. In addition, the preparations according to the invention impart loose body and hold to the hairstyle, without being sticky.

Accordingly the preparations according to the invention can, depending on their composition, be used, for example, as skin protection emulsion, cleansing milk, sunscreen lotion, nourishing lotion, day or night emulsion etc.

The microemulsions according to the invention also advantageously contribute to skin smoothing, particularly if they are provided with one or more substances which promote skin smoothing.

Where appropriate, it is possible and advantageous to use the preparations according to the invention as a base for pharmaceutical formulations. Corresponding requirements apply mutatis mutandis to the formulation of medicinal preparations. The transitions between pure cosmetics and pure pharmaceuticals are fluid in this connection. According to the invention, suitable pharmaceutical active ingredients are in principle all classes of active ingredient, preference being given to lipophilic active ingredients. Examples are: antihistamines, antiphlogistics, antibiotics, antimykotics, active ingredients which promote circulation, keratolytics, hormones, steroids, vitamins etc.

The cosmetic and dermatological preparations according to the invention can comprise cosmetic auxiliaries as are customarily used in such preparations, e.g. preservatives, bactericides, virucides, perfumes, antifoams, dyes, pigments which have a coloring action, thickeners, surface-active substances, emulsifiers, emollients, moisturizers and/or humectants, antiinflammatory substances, medicaments, fats, oils, waxes or other customary constituents of a cosmetic or dermatological formulation, such as alcohols, polyols, polymers, foam stabilizers, electrolytes, organic solvents.

Mixtures of the abovementioned solvents are used particularly advantageously.

Other constituents which may be used are fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids, alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

Unless stated otherwise, all amounts, percentages or parts refer to the weight of the preparations or of the respective mixture.

The examples below serve to illustrate the present invention.

The lecithin used in the examples below is Phospholipon 90 (Rhône-Poulenc, FR).

Instead of the ethoxylated sorbitan esters, it is also possible, for example, to use in each case PEG-50 hydrogenated castor oil isostearate with equal success.

### EXAMPLE 1

Face tonic

| | % by wt. |
|---|---|
| Lecithin | 0.5% |
| PEG-20 sorbitan isostearate | 2.5% |
| Glycerol isostearate | 0.5% |
| Glycerol | 5.000 |
| Cetearyl isononanoate | 2.500 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 2

Face tonic

| | % by wt. |
|---|---|
| Lecithin | 0.5% |
| PEG-20 sorbitan isostearate | 2.5% |
| Sorbitan isostearate | 0.5% |
| Glycerol | 5.000 |
| Cetearyl isononanoate | 2.500 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 3

Face tonic

| | % by wt. |
|---|---|
| Lecithin | 0.5% |
| PEG-20 sorbitan isostearate | 2.5% |
| Steareth-2 | 0.5% |
| Glycerol | 5.000 |
| Cetearyl isononanoate | 2.500 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 4

Face tonic

| | % by wt. |
|---|---|
| Lecithin | 1.0% |
| PEG-20 sorbitan isostearate | 2.5% |
| Phenylbenzimidazolesulfonic acid | 3.0% |
| Sodium hydroxide | 1.0% |
| Glycerol | 5.000 |
| Cetearyl isononanoate | 2.500 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 5

Face tonic

| | % by wt. |
|---|---|
| Lecithin | 0.5% |
| PEG-20 sorbitan isostearate | 2.5% |
| Glycerol isostearate | 0.5% |
| Glycerol | 5.000 |
| Cetearyl isononanoate | 2.500 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 6

Face tonic

| | % by wt. |
|---|---|
| Lecithin | 1.800 |
| PEG-50 hydrogenated castor oil isostearate | 5.200 |
| Glycerol | 5.000 |
| Dicaprylyl ether | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 7

Antiacne lotion

| | % by wt. |
|---|---|
| Lecithin | 3.000 |
| PEG-20 sorbitan isostearate | 4.000 |
| Glycerol | 5.000 |
| Dicaprylyl ether | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 8

Hair tonic

| | % by wt. |
|---|---|
| Lecithin | 3.000 |
| Oleth-15 | 4.000 |
| Glycerol | 5.000 |
| Dicaprylyl ether | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 9

Body lotion

| | % by wt. |
|---|---|
| Lecithin | 3.000 |
| PEG-45 palm kernel oil glycerides | 4.000 |
| Glycerol | 5.000 |
| Dicaprylyl ether | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 10

Base formulation for shaving foam

| | % by wt. |
|---|---|
| Lecithin | 3.000 |
| PEG-20 sorbitan monooleate | 4.000 |
| Glycerol | 5.000 |
| Dicaprylyl ether | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 11

Aftershave lotion

| | % by wt. |
|---|---|
| Lecithin | 1.000 |
| Polyglyceryl-10 stearate | 6.000 |
| Glycerol | 5.000 |
| Dicaprylyl ether | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 12

Face-cleansing lotion

| | % by wt. |
|---|---|
| Lecithin | 2.000 |
| Decaglyceryl monolaurate | 5.000 |
| Glycerol | 5.000 |
| Dicaprylyl ether | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 13

Shower oil, low foaming

| | % by wt. |
|---|---|
| Lecithin | 3.500 |
| PEG-20 glyceryl laurate | 3.500 |
| Glycerol | 5.000 |
| Dicaprylyl ether | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 14

Pump atomizer

| | % by wt. |
|---|---|
| Lecithin | 3.000 |
| PEG-20 monostearate | 4.000 |
| Glycerol | 5.000 |
| Dicaprylyl ether | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion. EXAMPLE 15

Transparent cleansing emulsion for greasy skin

| | % by wt. |
|---|---|
| Lecithin | 3.000 |
| PEG-20 glyceryl stearate | 4.000 |
| Glycerol | 5.000 |
| Dicaprylyl ether | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 16

Refreshing preshave lotion

| | % by wt. |
|---|---|
| Lecithin | 4.000 |
| Ceteraeth-12 | 3.000 |
| Glycerol | 5.000 |
| Dicaprylyl ether | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 17

Make-up removing lotion

| | % by wt. |
|---|---|
| Lecithin | 2.000 |
| PEG-20 sorbitan isostearate | 5.000 |
| Glycerol | 5.000 |
| Octyldodecanol | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 18

EXAMPLE 18
Base formulation for solubilizing perfume odorants
(perfume atomizer)

| | % by wt. |
|---|---|
| Lecithin | 2.000 |
| PEG-20 sorbitan isostearate | 5.000 |
| Glycerol | 5.000 |
| Cetearyl isononanoate | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 19

EXAMPLE 19
Base formulation for treating the scalp

| | % by wt. |
|---|---|
| Lecithin | 2.000 |
| PEG-20 sorbitan isostearate | 5.000 |
| Glycerol | 5.000 |
| Dioctylcyclohexane | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 20

| | EXAMPLE 20 |
|---|---|
| | % by wt. |
| Lecithin | 1.000 |
| Polyglyceryl-10 stearate | 6.000 |
| Glycerol | 5.000 |
| Dioctylcyclohexane | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 21

EXAMPLE 21
Deodorant/antiperspirant pump atomizer

| | % by wt. |
|---|---|
| Lecithin | 1.000 |
| PEG-20 sorbitan isostearate | 2.500 |
| Glycerol | 5.000 |
| Octyl dodecanol | 2.500 |
| Aluminum chlorhydrate | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 22

| | EXAMPLE 22 |
|---|---|
| | % by wt. |
| Lecithin | 2.000 |
| Oleth-15 | 5.000 |
| Glycerol | 5.000 |
| Caprylic/capric triglycerides | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 23

| | EXAMPLE 23 |
|---|---|
| | % by wt. |
| Lecithin | 3.000 |
| PEG-45 palm kernel oil glycerides | 4.000 |
| Glycerol | 5.000 |
| Dioctylcyclohexane | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 24

| | EXAMPLE 24 |
|---|---|
| | % by wt. |
| Lecithin | 3.000 |
| PEG-45 palm kernel oil glycerides | 4.000 |
| Glycerol | 5.000 |
| Cetearyl isononanoate | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 25

| | EXAMPLE 25 |
|---|---|
| | % by wt. |
| Lecithin | 2.000 |
| PEG-20 sorbitan monooleate | 5.000 |
| Glycerol | 5.000 |
| Octyl dodecanol | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 26

| | EXAMPLE 26 |
|---|---|
| | % by wt. |
| Lecithin | 2.000 |
| PEG-20 sorbitan monooleate | 5.000 |
| Glycerol | 5.000 |
| Caprylic/capric triglycerides | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 27

| | EXAMPLE 27 |
|---|---|
| | % by wt. |
| Lecithin | 2.000 |
| PEG-20 sorbitan monooleate | 5.000 |
| Glycerol | 5.000 |
| Cetearyl isononanoate | 5.000 |
| Preservative | q.s. |
| Water | ad |
| | 100.000 |

The oil phase and the water phase are each heated separately to 70-75° C. The water phase is added dropwise to the oil phase, and a gel forms. The further dropwise addition of the water phase and cooling of the mixture produces an O/W microemulsion.

### EXAMPLE 28

Shower oil

| | % by wt. |
|---|---|
| Lecithin | 0.250 |
| Lauryl ether sulfate (25%) | 40.000 |
| Glyceryl monolinoleate | 0.250 |
| Glycerol | 5.000 |
| Dicaprylyl ether | 3.000 |
| Sodium chloride | 7.500 |
| Water | ad |
| | 100.000 |

The oil phase and part of the water phase are each heated separately to 70-75° C. The water phase is added dropwise to the oil phase, and a gel forms. The further dropwise addition of the water phase and cooling of the mixture produces an O/W microemulsion.

### EXAMPLE 29

Face-cleansing gel

| | % by wt. |
|---|---|
| Lecithin | 6.660 |
| PEG-50 hydrogenated castor oil isostearate | 19.260 |
| Glycerol | 18.520 |
| Dicaprylyl ether | 18.520 |
| Water | 37.040 |

The oil phase and the water phase are each heated separately to 70-75° C. The water phase is added dropwise to the oil phase, and a gel forms.

### EXAMPLE 30

Face-cleansing gel

| | % by wt. |
|---|---|
| Lecithin | 6.660 |
| PEG-50 hydrogenated castor oil isostearate | 19.260 |
| Glycerol | 18.520 |
| Dicaprylyl ether | 18.520 |
| Water | 37.040 |

The oil phase and the water phase are each heated separately to 70-75° C. The water phase is added dropwise to the oil phase, and a gel forms.

### EXAMPLE 31

Eye make-up remover gel

| | % by wt. |
|---|---|
| Lecithin | 3.700 |
| Polyglyceryl-10 stearate | 22.200 |
| Glycerol | 18.500 |
| Dioctylcyclohexane | 18.500 |
| Water | 37.010 |

The oil phase and the water phase are each heated separately to 70-75° C. The water phase is added dropwise to the oil phase, and a gel forms.

### EXAMPLE 32

Hair gel

| | % by wt. |
|---|---|
| Lecithin | 11.100 |
| PEG-20 sorbitan isostearate | 14.800 |
| Glycerol | 18.500 |
| Dicaprylyl ether | 18.500 |
| Water | 37.100 |

The oil phase and the water phase are each heated separately to 70-75° C. The water phase is added dropwise to the oil phase, and a gel forms.

### EXAMPLE 33

Shower gel

| | % by wt. |
|---|---|
| Lecithin | 0.870 |
| Lauryl ether sulfate (25%) | 69.600 |
| Glycerol | 8.600 |
| Dicaprylyl ether | 8.700 |
| Sodium chloride | 12.230 |

The oil phase and the water phase are each heated separately to 70-75° C. The water phase is added dropwise to the oil phase, and a gel forms.

### EXAMPLE 34

Gel

| | % by wt. |
|---|---|
| Lecithin | 2.000 |
| Polyclycerol-10 stearate | 24.000 |
| Glycerol isostearate | 2.000 |
| Glycerol | 20.000 |
| Dioctylcyclohexane | 20.000 |
| Water | 32.000 |

1. A gel or low-viscosity transparent or translucent microemulsion of the oil-in-water type, comprising a water phase and an oil phase, which are essentially composed of low-volatility constituents, comprising: at least one phospholipid and at least one oil-in-water emulsifier and optionally at least one W/O emulsifier, obtainable by adding the water phase with its constituents to the oil phase with its constituents, in particular the phospholipid and the O/W emulsifier and optionally W/O emulsifier, the phases being mixed with one another and a gel state being achieved, and if a low-viscosity O/W microemulsion is desired, further parts of the water phase are added and the phases are mixed, it being possible, if desired, for the phases to comprise further auxiliaries, additives and/or active ingredients.

2. A process for the preparation of gels or low-viscosity transparent or translucent microemulsions of the oil-in-water type, comprising a water phase and an oil phase, which is essentially composed of low-volatility constituents, comprising at least one phospholipid and at least one oil-in-water emulsifier and optionally at least one W/O emulsifier, characterized in that a phospholipid is dissolved in the oil phase, optionally with further constituents, and the water phase, optionally with further constituents, is added thereto and the phases are mixed, during which the viscosity increases and, for example, the gels are obtained and, upon the further addition of the water phase, the microemulsions arise, where the oil-in-water emulsifier and optionally the W/O emulsifier can be added to the oil phase or can be added at the gel formation stage or else following preparation of the gels.

3. The gel as claimed in claim 1 or 2, characterized in that it is used as hair gel, shower gel, or skin gel.

4. The gel or microemulsion as claimed in claim 1 or 2, characterized in that it comprises deodorants or antiperspirants.

5. The gel or microemulsion as claimed in claim 1 or 2, characterized in that it comprises a UVA and/or UVB filter substance.

6. The gel or microemulsion as claimed in claim 1 or 2, characterized in that it comprises antioxidants.

7. The gel or microemulsion as claimed in claim 1 or 2, characterized in that it is used as a cosmetic cleansing preparation.

8. The gel or microemulsion as claimed in claim 1 or 2, characterized in that it is used for haircare.

9. The gel or microemulsion as claimed in claim 1 or 2, characterized in that it comprises active ingredients, additives or auxiliaries.
